(19) United States
(12) Patent Application Publication
(10) Pub. No.: US 2024/0041784 A1
(43) Pub. Date: Feb. 8, 2024

(54) MICROPARTICLES FOR MAGNETIC HYPERTHERMIA

(71) Applicant: LIQUIDS RESEARCH LIMITED, Bangor (GB)

(72) Inventor: Kevin O'GRADY, Bangor (GB)

(21) Appl. No.: 18/222,626

(22) Filed: Jul. 17, 2023

(30) Foreign Application Priority Data
Aug. 2, 2022 (GB) 2211256.9

(51) Int. Cl. A61K 9/50; A61K 41/00; A61N 1/40

(52) U.S. Cl. CPC A61K 9/5094; A61K 41/0052; A61N 1/406

(57) ABSTRACT

There is provided a microparticle comprising magnetic nanoparticles within a matrix such as a polymer sphere, wherein the magnetic nanoparticles have an anisotropy constant K in the range 1.0 to 3.0×10⁵ ergs cm⁻³ and exhibit a hysteresis loop under an alternating magnetic field so as to generate hysteresis heating whilst fixed within the polymer sphere. The alternating magnetic field has a maximum field strength in the range 100 to 400 Oe and a frequency in the range 25 kHz to 500 kHz. The magnetic nanoparticles have an axial ratio of at least 1.1 and are preferably magnetite and substantially crystalline. A method of synthesising such magnetic nanoparticles is also provided.

### FIELD OF THE INVENTION

This invention relates to magnetic microparticles for use in magnetic hyperthermia and a method of making such magnetic microparticles.

### BACKGROUND OF THE INVENTION

Magnetic hyperthermia occurs when magnetic nanoparticles suspended within a liquid and subjected to an AC magnetic field generate heat. Magnetic hyperthermia has been considered as a way to reduce tumours within the human body. However the degree of heating of magnetic particles in a body is unpredictable, in part due to the tendency of the magnetic particles to aggregate and physically rotate which can produce a larger than expected heating effect. It has not been possible to predict how many particles are required to act on a tumour, nor the frequency, field amplitude and the amount of time an AC magnetic field should be applied to generate an appropriate amount of heat for destruction of a defined quantity of diseased tissue or cells.

### SUMMARY OF THE INVENTION

In accordance with one aspect of the invention, there is provided a microparticle comprising magnetic nanoparticles within a matrix, wherein the magnetic nanoparticles have an anisotropy constant K in the range 1.0 to 3.0×10⁵ ergs cm⁻³ and exhibit a hysteresis loop under an alternating magnetic field. The microparticle is particularly suitable for use in magnetic hyperthermia treatment, the encapsulated magnetic nanoparticles are fixed in position and immobilised within the matrix, responsive to an applied magnetic field to generate heat due to magnetic hysteresis heating alone. This immobilisation prevents aggregation of the magnetic nanoparticles and allows the use of well dispersed, isolated nanoparticles for reproducibility of the heating effect. When the microparticles are placed in a liquid, such as blood, heating from stirring of the individual magnetic nanoparticles, or clusters of magnetic nanoparticles is prevented, which is important for providing a reproducible and predictable magnetic hyperthermia treatment.

The alternating magnetic field desirably has a maximum field strength in the range 100 to 400 Oe and a frequency in the range 25 kHz to 500 kHz, more preferably in the range 60 to 300 kHz.

The minimum median diameter of the magnetic nanoparticles may preferably be greater than 8 nm and more preferably greater than 12 nm. Preferably the magnetic nanoparticles have a median particle diameter of between 8 nm to 30 nm and desirably have a standard deviation of a log normal distribution volume between 0.2 and 0.5.

The magnetic nanoparticles preferably have an axial ratio of at least 1.1, and thus are spheroidal rather than spherical being elongated by greater than 10% along one axis as compared to an orthogonal axis.

The matrix may be an organic matrix such as provided by polymer or other plastics material, or a ceramic matrix or an inorganic matrix which may or may not be biodegradeable.

The matrix preferably contains 20 to 60 wt. % and more preferably 40 to 60 wt. % magnetic nanoparticles relative to the weight of the matrix material.

The magnetic nanoparticles preferably comprise at least one magnetic material formed from a divalent transition metal X, where X is one of the group of Fe, Co, Ni, Mn, Ba, Sr.

Desirably the magnetic nanoparticles are magnetite (Fe₃O₄) nanoparticles.

Preferably the magnetite nanoparticles are substantially crystalline and thus show a crystalline structure on a transmission electron microscope (TEM) micrograph.

Of preference the matrix is formed as a sphere, and more preferably as a polymer sphere, preferably having a diameter in the range 0.1 μm to 20 μm, and more preferably in the range 0.1 μm to 10 μm. The diameter of the sphere may be selected to ensure that in use the inverse of the relaxation time τ_{B} of the sphere is greater than an applied magnetic field frequency. The microparticle is thus sufficiently small to be injectable whilst being large enough not to exhibit rotation in response to an alternating magnetic field.

The matrix is preferably any suitable plastic material for sterile in vivo use and may be selected from one of the group consisting of polystyrene, poly(methyl methacrylate) or other biocompatible polymer such as poly(vinyl acetate), poly(vinyl chloride), divinylbenzene. The magnetic nanoparticles are preferably also sterile for in vivo use, and are typically magnetite nanoparticles.

In accordance with another aspect of the invention, there is provided a method of synthesising magnetic particles comprising disposing magnetic nanoparticles in an alkali solution, the magnetic nanoparticles comprising at least one magnetic material formed from a divalent transition metal X, where X is one of the group of Fe, Co, Ni, Mn, Ba, Sr, gradually adding an X II salt solution to create a growth mixture, heating the growth mixture and cooling to create modified magnetic nanoparticles with an anisotropy constant K in the range 1.0 to 3.0×10⁵ ergs cm⁻³.

The magnetic nanoparticles are preferably exposed to the growth mixture until the magnetic nanoparticles grow to reach an axial ratio of at least 1.1, so as to create elongate particles.

The growth mixture may be heated to around 100° C. for around 1 hour.

Desirably the method further comprises encapsulating the modified magnetic nanoparticles into microparticles, preferably polymer microspheres.

Preferably the magnetic nanoparticles are magnetite nanoparticles and the salt solution is an Fe II salt solution.

The invention will now be described, by way of example, with reference to the accompanying drawings in which:

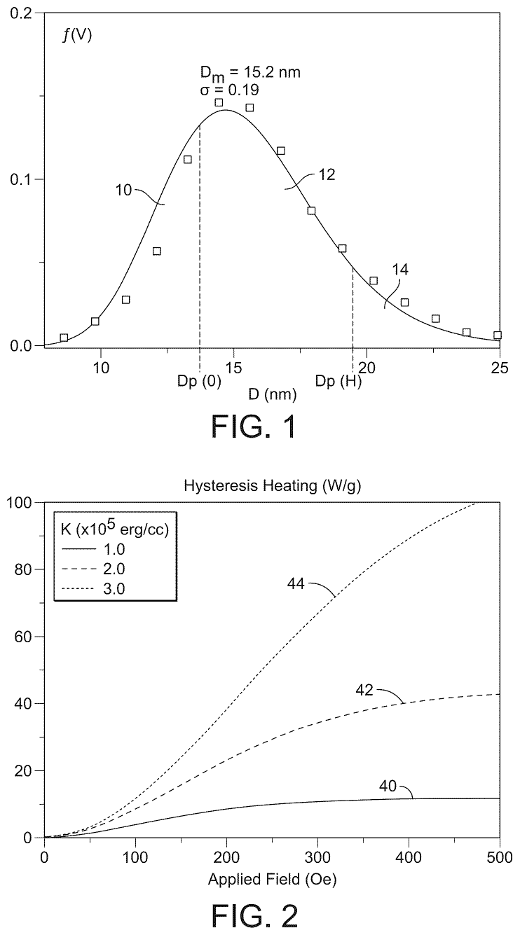

FIG. 1 is a graph illustrating contributions to heating under an applied magnetic field for magnetite nanoparticles in a colloid;

FIG. 2 is a graph illustrating hysteresis heating as maximum magnetic field and anisotropy constant are varied;

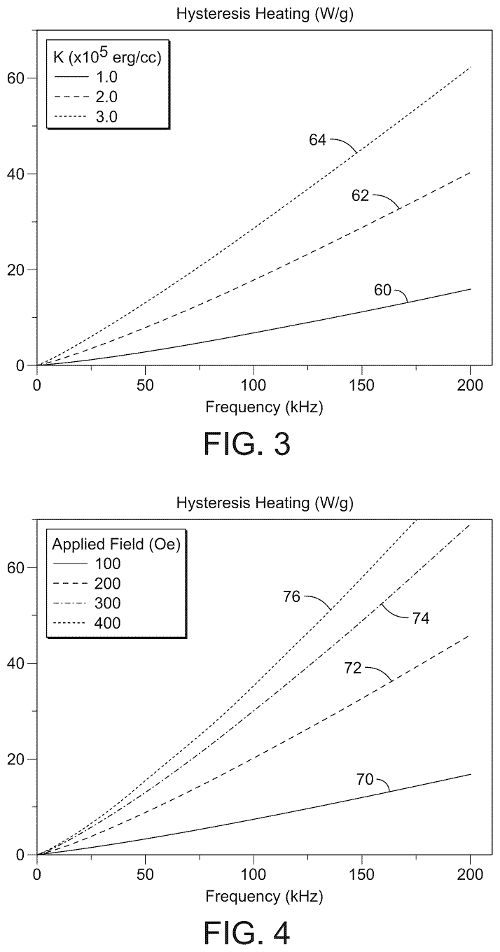

FIG. 3 is a graph illustrating hysteresis heating as field frequency and anisotropy constant are varied;

FIG. 4 is a graph illustrating hysteresis heating as maximum magnetic field and frequency are varied for a fixed anisotropy constant;

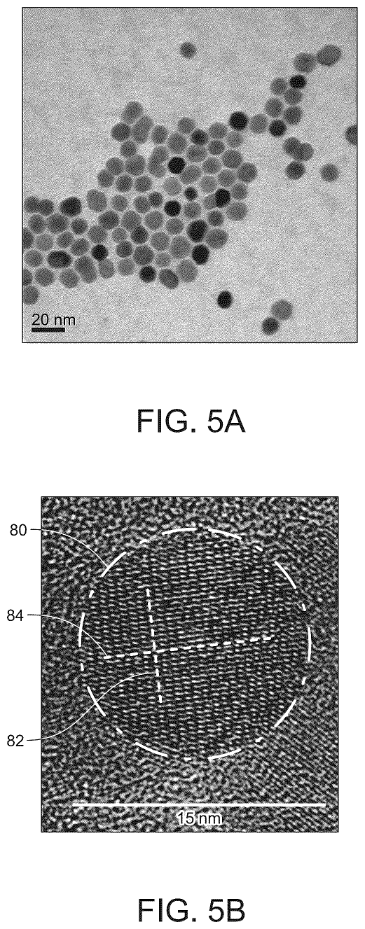

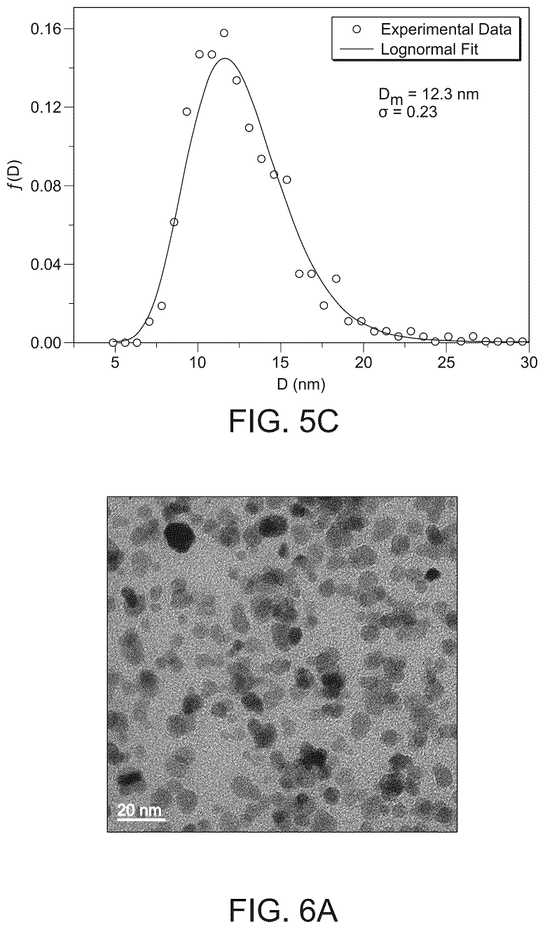

FIGS. 5(a) and (b) show TEM micrographs of modified magnetite nanoparticles made in accordance with the invention and FIG. 5(c) shows a graph illustrating their particle size distribution;

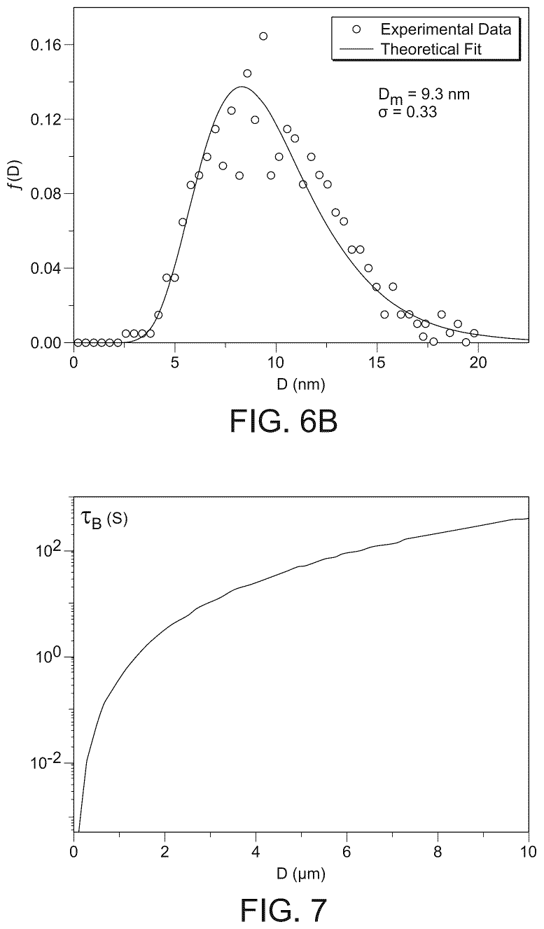

FIG. 6(a) shows TEM micrographs of prior art magnetite nanoparticles and FIG. 6(b) shows a graph illustrating their particle size distribution;

FIG. 7 shows a graph illustrating Brownian relaxation times for different particles;

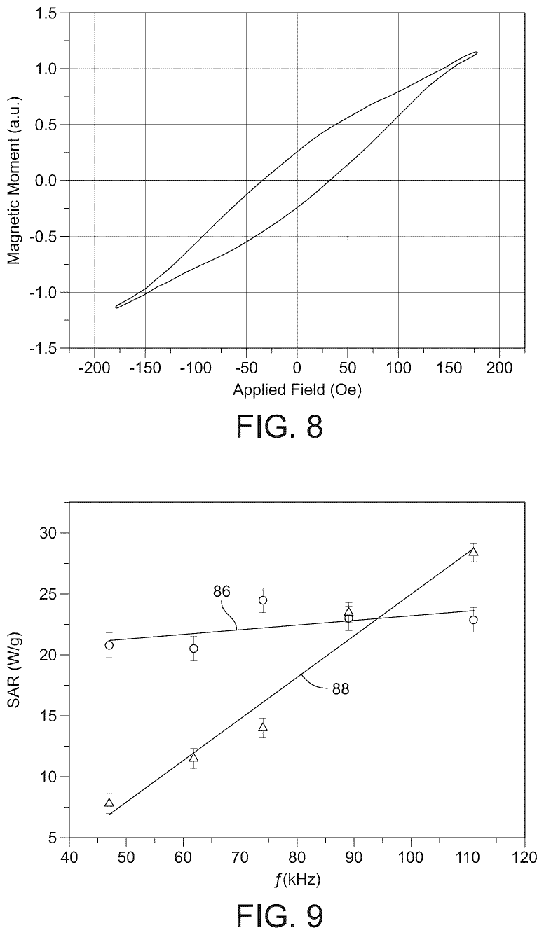

FIG. 8 shows the AC hysteresis loop at a frequency of 111.5 kHz and field amplitude of 100 Oe for modified magnetite nanoparticles in accordance with the invention when encapsulated in polymer microspheres of diameter of 0.3 μm; and

FIG. 9 shows a comparison of the SAR values for the same magnetic particles in water and when immobilised in polymer spheres in water.

### DESCRIPTION

When a colloid containing individual magnetic nanoparticles is exposed to an applied AC magnetic field, the magnetic nanoparticles dispersed in the liquid respond to the magnetic field to generate heating through susceptibility loss, hysteresis heating and also stirring as shown in FIG. 1 where the heating effects present for different diameter (D) magnetite Fe₃O₄ particles is shown for a temperature of 293 K at an applied field of 250 Oe and a field frequency of 111.5 kHz. Heating through susceptibility loss occurs in region 10 until above a certain diameter Dₚ(0) heating due to susceptibility losses will not occur, this diameter being given by:

$${D}_{p} ( 0 ) = {( \frac{6 {k}_{B} T ln ( {tf}_{0} )}{π K} )}^{1 / 3} \qquad Equation 1$$

where t is the time of measurement, kB Boltzmann's constant, T is the temperature of measurement, f is 10⁹ s⁻¹, and K is the anisotropy constant.

Heating through hysteresis losses occurs in region 12 and above a certain diameter Dₚ(H) which is dependent on the applied field amplitude, particles will not contribute to either susceptibility or hysteresis loss heating and only viscous heating due to stirring of the particles within the colloid will occur, region 14. This effect becomes more pronounced as particles aggregate and can lead to very high temperatures in the colloid. Dₚ(H) is given by:

$${D}_{p} ( H ) = {[ 1 - \frac{H {M}_{s}}{0.96 K} ]}^{- \frac{2}{3}} {D}_{p} ( 0 ) \qquad Equation 2$$

where Mₛ is the saturation magnetisation and H is the magnetic field amplitude.

For magnetite nanoparticles having a diameter Dₚ(0)<D<Dₚ(H), only hysteresis loss heating will occur arising from the energy released as a magnetic material is cycled around its hysteresis loop by an AC magnetic field.

For controllable magnetic hyperthermia, quantifiable and predictable heating is required which cannot be achieved from individual magnetic nanoparticles dispersed in a liquid, such as water or blood. By immobilising magnetic nanoparticles within microspheres, with the magnetite nanoparticles dispersed throughout a matrix forming the microsphere, the magnetic nanoparticles cannot move relative to one another under the influence of an alternating magnetic field. By fixing the magnetic nanoparticles in position, they cannot physically rotate and generate uncontrollable and unpredictable heat by viscous drag.

By selecting the size distribution of magnetic nanoparticles and immobilising the magnetic nanoparticles within a matrix, so as to only generate heating through hysteresis heating, the heating effect is reduced by approximately 40% over unconstrained magnetic nanoparticles in a liquid.

The amount of heat generated from hysteresis heating Pₕ is proportional to the frequency (f) multiplied by the area of the hysteresis loop, given by:

Pₕ=2Mₛf ∫ᵥ_{ₚ₍ₒ₎}^{v}^{_{p(H)}}H_{c}(V)f(V)dV Equation 3

where Vₚ(H) is the volume of a particle having a diameter Dₚ(H), H_{c} is the coercivity and f(V) is the distribution of physical volumes.

The anisotropy constant of the magnetic nanoparticles and the applied field affects the hysteresis heating. With conventional spherical magnetite nanoparticles, the anisotropy constant K is less than 1×10⁵ ergs cm⁻³ which results in minimal hysteresis heating regardless of the field applied. The modified magnetic nanoparticles of the present invention are formed with an elongated shape as discussed in detail below so as to have an increased anisotropy constant, typically with the anisotropy constant K in the range 1.0 to 3.0×10⁵ ergs cm⁻³. This greatly increases the hysteresis heating able to take place at applied fields of 100 to 400 Oe.

FIG. 2 shows a graph of hysteresis heating Pₕ for a set frequency of 111.5 kHz for a maximum applied field H set between 100 and 500 Oe for immobilised modified magnetite nanoparticles with three different anisotropy constants and having a median diameter 12.3 nm, standard deviation σ of the distribution of 0.23, Mₛ of 420 emu cm⁻³, at a temperature of 293K. Solid line 40 represents an anisotropy constant K of 1×10⁵ ergs cm⁻³, with dashed line 42 representing an anisotropy constant K of 2×10⁵ ergs cm⁻³. Dotted line 44 represents magnetite nanoparticles having an anisotropy constant of K of 3×10⁵ ergs cm⁻³. Lines 40, 42, 44 exhibit change with increasing field, with line 40 increasing until about 300 Oe before plateauing. It can be seen that the modified magnetite nanoparticles exhibit increased hysteresis heating as their anisotropy constant increases.

FIG. 3 shows the effect on hysteresis heating at 293 K when varying the frequency from 0 kHz to 200 kHz at a fixed applied field of 180 Oe for modified magnetite nanoparticles with different anisotropy constants. Solid line 60 represents an anisotropy constant K of 1×10⁵ ergs cm⁻³, with dashed line 62 representing an anisotropy constant K of 2×10⁵ ergs cm⁻³. Dotted line 64 represents modified magnetite nanoparticles having an anisotropy constant of K of 3×10⁵ ergs cm⁻³. All lines 62, 64 and 66 increase with frequency and demonstrate that for a given frequency the modified magnetite nanoparticles exhibit increased hysteresis heating as their anisotropy constant increases. Frequencies in the range 25 to 500 kHz, and more preferably in the range 150 to 300 kHz, are suitable for hyperthermia treatment.

Using such data allows the hysteresis heating generated to be calculated using equation 3 and controlled. The degree of hysteresis heating required can be configured by choosing particles with the required anisotropy constant and selecting the magnetic field strength and frequency known to give appropriate hysteresis heating for that anisotropy constant. A lower operating frequency provides a greater degree of control over the heating exhibited by the immobilised modified magnetite nanoparticles and by selecting a field amplitude in the range 150 Oe to 400 Oe substantially the whole of the magnetic particle size distribution will generate hysteresis heating. FIG. 4 shows how hysteresis heating alters for different applied fields at different frequencies for modified magnetite nanoparticles with an anisotropy constant of K of 3×10⁵ ergs cm⁻³. Solid line 70 shows the response at a field of 100 Oe, dashed line 72 at a field of 200 Oe, dotted and dashed line 74 at a field of 300 Oe and dotted line 76 at a field of 400 Oe.

The preparation of the modified elongate magnetite nanoparticles with an anisotropy constant

K in the range 1.0 to 3.0×10⁵ ergs cm⁻³ will now be described.

The standard method for the preparation of magnetite nanoparticles is the co-precipitation process of Khalafalla and Riemers, see U.S. Pat. No. 3,843,540. This gives nanoparticles of diameter 10 nm or less which are too small to give significant hysteresis. To produce magnetite nanoparticles with an increased anisotropy constant and which are suitable for magnetic hyperthermia via hysteresis, an alternative growth method has been derived.

Thus in accordance with one aspect of the invention, the preparation of suitable magnetite nanoparticles for magnetic hyperthermia proceeds in two stages. The first stage is the standard co-precipitation method for the production of magnetite nanoparticles of Khalafalla and Riemers as above. The detailed example below uses a combination of Iron II Chloride and Iron III Chloride salts dissolved in water. Those of skill in the art will also know that other combinations of Iron II and Iron III salts can also be used with the appropriate molar ratio. The chlorides dissolved in water are then combined with a concentrated solution of sodium hydroxide (NaOH) via a peristaltic pump. Those of relevant skill in the art will be aware that other hydroxides such as potassium hydroxide (KOH) or ammonia (NH₃OH) or any other suitable hydroxide can also be used. This co-precipitation process produces spherical magnetite nanoparticles with a median diameter ranging between 8 and 11 nm.

The second stage of preparation involves exposing the magnetite nanoparticles to a growth layer solution such as FeCl₂ to deposit additional Iron II onto the particles formed by the co-precipitation method. This increases the diameter of the particles with the growth process acting to elongate the particles so they are no longer spherical and become spheroid, so increasing their anisotropy constant. Following these two stages the modified elongate magnetite nanoparticles are then thoroughly washed and re-introduced to a solution of concentrated alkali where any of the alkalis above can be used. A solution of Iron II Chloride (FeCl₂) is then added at a constant rate with further stirring and heating for a period of one hour at 100° C. Other rates of addition and other similar heating regimes for varying times can be used. Those of skill in the art will also be aware that other Iron II compounds such as iron sulphate could also be used for this additional growth process.

### EXAMPLE

A detailed example of preparation of microparticles configured for hyperthermia using the two stage controlled growth process is as follows.

### First Stage: Co-Precipitation

An Iron III salt such as FeCl₃.6H₂) (142.6 g, 0.527 mol) was dissolved in distilled water giving a total volume of 400 ml. A second solution of an Iron II salt such as FeCl₂4H₂O (111.2 g, mol) was dissolved in distilled water giving a total volume of 400 ml. The two solutions were then combined and mixed with an overhead stirrer at 500 rpm for 5 minutes. A 10M NaOH aqueous solution (580 ml, 5.8 moles) was then added via peristaltic pump at a rate of 1700 ml/min to the Fe II/III salt solution producing a black precipitate.

Once the addition of the NaOH solution was completed stirring was continued for 10 minutes before the resulting magnetite nanoparticles were washed in water two times. Distilled water was added bringing the mass of magnetite slurry to 1000 g, the magnetite nanoparticles having a median diameter between 8 and 11 nm.

### Second Stage: Preparation of Growth Layer

To achieve deposition of a growth layer on the magnetite nanoparticles and so create modified magnetite nanoparticles, for this example with a median diameter of 12.3 nm, an alkali solution such as 10M NaOH aqueous solution (15 ml, 0.15 moles) was added to the magnetite slurry and pH checked to confirm pH =12. The slurry was stirred with an overhead stirrer at 500 rpm for 5 minutes.

To create a growth layer solution, an Iron II salt such as FeCl₂.4H₂O (12.92 g, 0.065 mol) was dissolved in distilled water giving a total volume of 65 ml (1.0 M solution). To achieve thicker growth layers, the concentration of FeCl₂ is increased and for example to achieve modified magnetite particles with a median diameter of 15.4 nm after growth, FeCl₂.4H₂O (51.69 g, 0.260 mol) was dissolved in distilled water giving a total volume of 260 ml (1.0 M solution).

Stirrer speed was reduced to 250 rpm and the growth layer FeCl₂ solution was added via peristaltic pump at a rate of 250 ml/min. Slurry was stirred at 250 rpm at room temperature for one hour before being heated to 100C and stirred for one hour. The resultant slurry was allowed to cool and washed with six times with tap water until pH=8. The magnetite nanoparticles within the slurry are now modified in shape being elongate with an axial ratio of at least 1.1 and typically in the range 1.1 to 2.2. The addition of the extra Fe II using the growth layer solution restores the Fe3+ rich magnetite particle surface back closer to the ideal Fe3+/Fe2+ ratio of 2:1. This increases the saturation magnetisation M s from about 70 emu/g to about 80 emu/g. As shape anisotropy constant varies with Mₛ², this increases K by about 30% with further increase in K due to elongation of the particles during the growth process.

Typically the median diameter of the modified magnetite nanoparticles is greater than 8 nm and less than or equal to 30 nm. The elongate magnetite nanoparticles resulting from the growth process have an anisotropy constant K in the range of 1.0 to 3.0×10⁵ ergs cm⁻³ so as to exhibit a hysteresis loop under an applied field in the range 100 Oe to 400 Oe. Small variations will occur in anisotropy constant within any batch of particles obtained from the growth process due to variation in particle size within any such batch as is shown in FIG. 5(c). Thus the anisotropy constant measured and observed is strictly speaking the average of the anisotropy constants of all magnetic particles assessed together in any single measurement.

The modified magnetite Fe₃O₄ particles produced by the controlled growth process are shown in FIGS. 5(a) and 5(b). FIG. 5(a) is a TEM micrograph showing the modified magnetite particles. The modified particles are crystalline and much larger and more elongate than those produced by co-precipitation alone. The particles are well dispersed as a result of the oleic acid being able to bond to the Fez growth layer formed during the second stage. The particles are elongated, being spheroid rather than spherical, and are highly crystalline as can be deduced from the 10 to 15% of particles showing black in the TEM micrograph indicating the particles meet the Bragg condition. The crystalline nature of the modified particle is confirmed by FIG. 5(b) which shows a TEM image of a single particle 80 as shown within the white—line. The regular crystalline structure can be seen, see in particular dashed lines 82, 84 which have been superimposed to aid the reader.

The diameter of the modified particles is measured using transmission electron microscopy and the modified particles have a median diameter in the range 8 nm to 30 nm with a standard deviation of a lognormal distribution volume of between 0.2 and 0.5 as shown in FIG. 5(c). For a diameter of 12.3 nm, the volume of these modified elongated particles is 86% larger than in standard 10 nm particles.

Unmodified magnetite nanoparticles are shown for comparison in FIG. 6(a) and it can be seen that the particles are chaotic within the TEM sample and are not well dispersed. The colour gradation indicates the magnetite is not crystalline and the size distribution as shown in FIG. 6(b) is much broader having standard deviation σ of the distribution of 0.33 rather than for the modified magnetite and thus the modified magnetite has a more consistent particle size distribution.

After the first two stages are completed, the modified particles are coated with oleic acid to enable the modified magnetite nanoparticles to be separated from the slurry. Thus water (300 ml) and 35% Ammonia solution (20 ml, 0.36 moles) are added to the slurry followed by oleic acid (30 ml). This coating mixture was stirred with an overhead stirrer at 600 rpm for one hour. M HCl was then added to the stirring suspension until pH=4.0 when the coated particles separate from the slurry and the coated modified magnetite particles washed.

The modified magnetite nanoparticles resulting from the growth process were then encapsulated into a matrix to create microparticles useable in hyperthermia. The matrix can be an organic matrix such as provided by polymer or other plastics material, or a ceramic matrix or an inorganic matrix which may or may not be biodegradeable. There are many methods for the production of microparticles or microbeads known to those of ordinary skill in the art. Here by way of example only, one such process is described.

To prepare polymer microspheres encapsulating modified magnetite nanoparticles, 7.5 g Crodafos 0.5 A with 35.0 g styrene monomer was mixed in a beaker with 2 g of modified magnetite particles synthesised using the growth process as aforesaid and then mixed with a rotor stator mixer for 15 minutes. The resulting mixture was then subjected to a high energy ultrasonic disintegration for a further 30 minutes to disperse particles using a Soniprep® 150. 30 g of the styrene-based ferrofluid was then added to the water phase (10% styrene in water) and dispersed using an ultrasonic disintegrator for 10 minutes to generate an emulsion. 0.3 g of water-soluble initiator, for example Potassium Persulphate K₂(SO₄)₂, was then added to the emulsion.

After this, the prepared emulsion was then loaded into a 500 ml three necked flask fitted with stirrer, condenser and nitrogen inlet for polymerisation (step 1). The emulsion was stirred at rpm for 5 mins under a flow of nitrogen (step 2). The temperature was then raised to 70° C. and kept at 70° C. under a flow of nitrogen for 6 hours (step 3), with the solution was allowed to cool under a flow of nitrogen (step 4).

The solution was then added to a 2.5 litre stainless steel beaker and placed over a magnetic plate to achieve separation (step 5). The solution was topped up to 1 litre with 0.5 wt % sodium dodecyl sulphate, stirred and re-separated on a magnet (step 6). The supernatant liquid was decanted (step 7). The remaining polymer microspheres containing the modified magnetite nanoparticles were then washed several times in deionised water.

Thus elongate magnetite nanoparticles with an anisotropy constant K in the range 1.0 to 3.0×10⁵ ergs cm⁻³ are encapsulated into a matrix, typically a plastics material matrix achieved by emulsion polymerisation, to create polymer microspheres sterile for in vivo use which range in size from 0.1 μm to 20 μm, and more preferably from 0.1 μm to 10 μm. The volumetric loading of the magnetite within the polymer spheres is typically in the range 20 to 60% by weight, and more preferably 40 to 60% by weight, and is sufficient to ensure the microparticles exhibit hysteresis heating. The polymer microspheres can be functionalised with external markers so as to attach to cancer cells. In addition to prove the efficiency of the microspheres in tumour reduction, the surface of the microspheres can be functionalised with drugs such as doxyrubicin or cancer cell inhibitors such as the synthetic peptide Nucant.

The resulting microspheres are of further benefit as such spheres, having a diameter in the range of 0.1 μm to 20 μm encapsulating magnetic nanoparticles with an anisotropy constant K in the range 1.0 to 3.0×10⁵ ergs cm⁻³ so as to exhibit a hysteresis loop under an alternating magnetic field, have a Brownian relaxation time sufficient to ensure that individual microspheres cannot rotate in an AC field even when magnetic nanoparticles within the sphere are fully magnetised. This ensures that any heating is from hysteresis heating alone without any residual effects from movement or clustering of the microspheres.

The rotation time of a microsphere in a liquid is determined by the Brownian relaxation time (τ_{B}) given by

(τ_{B}=3V_{H}η/k_{B}T) Equation 4

where V_{H} is the hydrodynamic volume of the particle, η the viscosity of the liquid environment, kB is Boltzmann's constant and T is the temperature. By encapsulating the individual magnetic particles within a polymer sphere that is much larger than the magnetic particles, the time for the polymer sphere to rotate as the magnetic field changes direction becomes much greater than the switching time of the AC magnetic field. Stirring by movement of the microparticles themselves cannot take place in response to the alternating magnetic field. This is shown in FIG. 7 for particles of varying diameter D. Where D is 0.1 μm, the Brownian relaxation time is around 0.4 ms which means the particle is unable to rotate within the switching time of the AC magnetic field as the inverse of the relaxation time τ_{B} of the sphere is greater than the applied magnetic field frequency. For a microparticle with D of 10 μm, then τ_{B} is around 400 ms.

As the magnetic nanoparticles are encapsulated within a larger micro-sized particle, namely the polymer microsphere itself, heating due to rotation and thus stirring by the microsphere is eliminated as the microsphere itself has no permanent net magnetic moment and the rotation time for such a microsphere is greater than the switching time of the alternating magnetic field.

FIG. 8 shows the hysteresis loop in water for polymer spheres encapsulating modified magnetite nanoparticles, the spheres having an diameter of 0.31 μm and the field having a frequency 111 kHz and field amplitude of 180 Oe. The heat generated by magnetic nanoparticles exposed to an AC field is generally characterised by a parameter known as the Specific Absorption Ratio (SAR) given by

$$S A R = \frac{C ρ}{ϕ} \frac{Δ T}{Δ t} \qquad Equation 5$$

where C is the specific heat of the colloid, ρ is its density and ϕ is the concentration of elemental iron (Fe) in the colloid. AT is the rise in temperature in a time Δt. Measuring the SAR of the magnetite nanoparticles when encapsulated in polymer spheres gave a SAR for hysteresis heating of 28.4 W/g.

FIG. 9 shows that hysteresis heating from magnetite particles encapsulated within polymer spheres, see line 88, is lower than that for non-encapsulated magnetite particles in water, see line 86, at low frequencies but for f>100 kHz the polymer spheres exhibit a higher SAR. This shows that using defined AC field conditions, effective and controlled heating can be achieved.

Thus magnetic nanoparticles can be synthesised using a growth process as aforesaid to have an anisotropy constant K of between 1.0 to 3.0×10⁵ ergs cm⁻³. When encapsulated in polymer microspheres and exposed to a magnetic field in the range of 100 to 400 Oe and a field frequency in the range of 25 kHz to 500 kHz, localised hysteresis heating can be achieved, with such magnetic hyperthermia useful for treating tumours.

1. A microparticle comprising magnetic nanoparticles within a matrix, wherein the magnetic nanoparticles have an anisotropy constant K in the range 1.0 to 3.0×10⁵ ergs cm⁻³ and exhibit a hysteresis loop under an alternating magnetic field.

2. A microparticle according to claim 1, wherein the alternating magnetic field has a maximum field strength in the range 100 to 400 Oe.

3. A microparticle according to claim 1, wherein the alternating magnetic field has a frequency in the range 25 kHz to 500 kHz.

4. A microparticle according to claim 1, wherein the magnetic nanoparticles have a median diameter between 8 to 30 nm.

5. A microparticle according to claim 1, wherein the magnetic nanoparticles have a standard deviation of a log normal distribution volume between 0.2 and 0.5.

6. A microparticle according to claim 1, wherein the magnetic nanoparticles have an axial ratio of at least 1.1.

7. A microparticle according to claim 1, wherein the matrix is an organic matrix, or a ceramic matrix or an inorganic matrix.

8. A microparticle according to claim 1, wherein the matrix contains 20 to 60 wt. % and more preferably 40 to 60 wt. % magnetic nanoparticles relative to the weight of the matrix.

9. A microparticle according to claim 1, wherein the magnetic nanoparticles comprise a magnetic material formed from at least one divalent transition metal X, where X is one of the group of Fe, Co, Ni, Mn, Ba, Sr.

10. A microparticle according to claim 1, wherein the magnetic nanoparticles are magnetite nanoparticles.

11. A microparticle according to claim 10, wherein the magnetite nanoparticles are substantially crystalline.

12. A microparticle according to claim 1, wherein the matrix is formed as a sphere, preferably having a diameter in the range 0.1 μm to 20 μm, and more preferably in the range 0.1 μm to 10 μm.

13. A microparticle according to claim 12, wherein the diameter of the sphere is selected to ensure that in use the inverse of the relaxation time τ_{B} of the sphere is greater than an applied magnetic field frequency.

14. A microparticle according to claim 1, wherein the matrix is a polymer sphere.

15. A microparticle according to claim 14, wherein the polymer sphere is formed from one of the group consisting of polystyrene, poly(methyl methacrylate), or any biocompatible polymer such as poly(vinyl acetate), poly(vinyl chloride), divinylbenzene.

16. A method of synthesising magnetic particles comprising disposing magnetic nanoparticles comprising a magnetic material formed from at least one divalent transition metal X, where X is one of the group of Fe, Co, Ni, Mn, Ba, Sr in an alkali solution, gradually adding an X II salt solution to create a growth mixture, heating the growth mixture and cooling to create modified magnetic nanoparticles with an anisotropy constant K in the range 1.0 to 3.0×10⁵ ergs cm⁻³.

17. A method of synthesising magnetic particles according to claim 16, wherein the magnetic nanoparticles are exposed to the growth mixture until the magnetic nanoparticles grow to reach an axial ratio of at least 1.1.

18. A method of synthesising magnetic particles according to claim 16, wherein the growth mixture is heated to around 100° C. for around 1 hour.

19. A method of synthesising magnetic particles according to claim 16, wherein the modified magnetic nanoparticles are encapsulated into polymer microspheres.

20. A method of synthesising magnetic particles according to claim 16, wherein the magnetic nanoparticles are magnetite nanoparticles and the salt solution is an Fe II salt solution.

21. Magnetic particles as formed in accordance with claim 16.
